(19) United States
(12) Patent Application Publication
(10) Pub. No.: US 2003/0232099 A1
(43) Pub. Date: Dec. 18, 2003

(54) Health-care products and methods for preparing and using the same

(75) Inventors: Ling Pan, Kunming (CN); Deliang Li, Kunming (CN); Liqun Zhang, Kunming (CN); Wenxu Tang, Kunming (CN); Chengjin Yang, Kunming (CN); Chongkun Gao, Kunming (CN); You Feng, Kunming (CN); Mian Chen, Yunnan (CN)

Correspondence Address: AKIN GUMP STRAUSS HAUER & FELD L.L.P., ONE COMMERCE SQUARE, 2005 MARKET STREET, SUITE 2200, PHILADELPHIA, PA, 19103-7013, US

(73) Assignee: Yunnan Baiyao Group Co., Ltd.

(21) Appl. No.: 10/454,307

(22) Filed: Jun. 4, 2003

(30) Foreign Application Priority Data
Jun. 7, 2002 (CN) 02113847.8

(51) Int. Cl. A61K 35/78

(52) U.S. Cl. 424/757; 424/773

(57) ABSTRACT

A multifunction health-care product and methods for preparing and using the same are provided. The health-care product contains a powder of lotus root joints and/or an extract of the joints as the main active ingredient. A preferred composition of the product contains 4-7.5 parts by weight extract of the joints, 2-15 parts by weight powder of lotus root joints, 0.08 to 2 parts by weight green tea and/or an extract thereof, and 0.08 to 0.5 parts by weight notoginseng and/or an extract thereof. The method for the preparation of the composition involves directly pulverizing the lotus root joints, putting the pulverized lotus root joints in product-grade solvent for extraction, and then filtering it to obtain a filtrate solution which is the extract of the joints. The health-care product can effectively improve IR, thus improving and preventing type-II diabetes mellitus, hypertension, hyperlipoidemia, and cardio-cerebral vascular diseases caused by being obese, and also has the effect of effecting weight loss.

### BACKGROUND OF THE INVENTION

The present invention pertains to health-care products, especially to health-care products capable of improving insulin resistance (IR), and to methods for their preparation and use.

Along with the continuous improvement of living standard and the change of lifestyle, the number of people suffering from obesity, type-II Diabetes Mellitus, hypertension, hyperlipoidemia and cardio-cerebral vascular diseases have continuously increased, and these diseases often appear with complications. Due to high lethality and disability rates, these diseases represent important ones which presently threaten human health. In recent decades, many domestic and foreign studies have indicated that these diseases have the same pathogenetic basis, i.e., Insulin Resistance. IR is the key cause of these diseases, and obesity is the prelude of other diseases. IR means that a normal or higher insulin (Ins) level is contained in the blood of a human body, but it could just perform an insufficient biological effect, i.e., the Ins-sensitivity and Ins-reactivity of the targeted tissue and organs (muscle, liver and fat tissue, etc.) of organic Ins have been decreased. To overcome IR, B islet cells have to secrete more Ins, thus causing compensative high hyperinsulinemia (HI), which may in turn further induce or aggravate these diseases. At present, modern medicine has turned its efforts to improving IR for better treating these diseases.

At present, the medicines for improving IR are mainly chemically synthesized substances (referred to as western medicine), such as insulin sensitizers, thiazolidine diketone derivatives (TZD) for the treatment of type-II Diabetes Mellitus. However, since the duration of clinical applications of these types of medicines has not been long enough, their long-term adverse effects still need to be examined. Accordingly, these medicines seem to be unsuitable for long term administration. Until now, there is still no instant Chinese herbal medicine or health-care product that shows definite improvements and/or treatment effects on IR.

IR has a complicated pathogenesis which involves deficiencies on different levels and links in the Ins pre-receptor, receptor and post-receptor. So far, there is still a strong demand for a safe, effective medicine that could comprehensively improve IR and related diseases.

### BRIEF SUMMARY OF THE INVENTION

A health-care product is provided, which comprises at least one of a powder and an extract of lotus root joints.

A method for preparing a health-care product, comprises the steps of:

a) Pulverizing lotus root joints to obtain a powder of the joints;

b) Putting the powder of the joints in a product-grade solvent to form a mixture for extraction, filtering the mixture to form a filtrate solution, and using the filtrate solution as an extract of the joints; and

c) Blending at least one conventional product-grade adjuvant with at least one of the powder of the joints and the extract of the joints, and formulating the blend into a form selected from the group consisting of a tablet, a capsule, a soluble granule, a solution and an injection.

Finally, a method is provided for preparing a medicine selected from the group consisting of a medicine for improving human insulin resistance; a medicine for treating or preventing obesity; a medicine for treating or preventing hypertension; a medicine for treating or preventing hyperlipoidemia; a medicine for treating or preventing diabetes mellitus; a medicine for improving blood viscosity, preventing thrombosis, and promoting microcirculation; and a medicine for treating or preventing Alzheimer's disease and senility. The method comprises using the above-described health care product to form a medicine.

### BRIEF DESCRIPTION OF THE SEVERAL VIEWS OF THE DRAWINGS

The foregoing summary, as well as the following detailed description of the invention, will be better understood when read in conjunction with the appended drawing. For the purpose of illustrating the invention, there is shown in the drawing an embodiment which is presently preferred. It should be understood, however, that the invention is not limited to the precise arrangements and instrumentalities shown.

In the drawing:

FIG. 1 shows a graph of change in weight for rats in different groups in a trial.

### DETAILED DESCRIPTION OF THE INVENTION

To overcome the deficiency in existing technology, the present invention provides a multifunction health-care product, which could improve IR effectively and prevent the onset of type-II diabetes mellitus, hypertension, hyperlipoidemia and cardio-cerebral vascular disease caused by obesity without adverse effects. Further, the existing symptoms caused by these diseases could also be substantially improved. Finally, the multifunction health care product provides a good anti-obesity effect.

According to one aspect of the invention, there is provided a multifunction health-care product comprising a powder or an extract of lotus root joints.

In one preferred embodiment, the health-care product preferably further comprises green tea and/or its extract, notoginseng and/or its extract, or a mixture of the two. A preferred composition of the health care product comprises about 4 to 30 parts by weight, more preferably about 5 to 15 parts by weight, and most preferably about 4 to 7.5 parts by weight extract of lotus root joints; about 0.08 to 2 parts by weight green tea and/or its extract, and about 0.08 to 0.5 parts by weight notoginseng and/or its extract. In addition, the health-care product may further comprise about 4 to 15 parts by weight of a powder of lotus root joints.

The health-care product described above may be formulated into conventional administration forms, such as a tablet, capsule, soluble granule, solution, injection, etc.

According to another aspect of the invention, a method for preparing a health care product is provided. The method comprises the following steps:

a) Pulverizing lotus root joints to obtain a powder of the joints;

b) Putting the powder of the joints in a product-grade solvent for extraction, filtering the solution to yield a filtrate solution, and using the filtrate solution as the extract of the joints; and

c) Blending at least one conventional product-grade adjuvant with the powder of the joints or the extract of the joints, and formulating the blend into a form of a tablet, capsule, soluble granule, solution and/or injection.

The product-grade solvent is preferably a conventional food-grade solvent, more preferably fresh water or ethanol.

Steps a) and b) may be carried out by any of the following variants:

(1) Drying lotus root joints and pulverizing the dried joints into a powder having a granule size of about 20 mesh; adding to the powder about 5 to 10 times by weight of about 30%˜90% ethanol based on the weight of the powder, to form a mixture; extracting the mixture at room temperature for 24 hours and then filtering the mixture to produce a filtrate solution and a residue; repeating the extraction step an additional two times; combining the filtrate solutions; and concentrating the combined filtrate solutions under vacuum and freeze-drying the solution into a dried powder; or

(2) Drying lotus root joints and pulverizing the dried joints into a powder having a granule size of about 20 mesh; adding to the powder about 5 to 10 times by weight of about 30%˜90% ethanol based on the weight of the powder, to form a mixture; heating the mixture in a water bath, refluxing for about 30 to 60 minutes, and filtering the mixture while it is hot to form a filtrate solution and a residue; repeating the heating and filtering steps an additional two times; combining the filtrate solutions; concentrating the solutions at a low temperature under reduced pressure; and freeze-drying the solutions into a dried powder; or

(3) Drying lotus root joints and pulverizing the dried joints into granules having a size of a soybean; adding to the granules about 6 to 10 times by weight of fresh water based on the weight of the granules, to form a mixture; heating the mixture at a boiling point for about 30 to 40 minutes and filtering the mixture while it is hot to form a filtrate solution and a residue; repeating the heating and filtering steps an additional two times; combining the filtrate solutions; concentrating the solutions under vacuum; and freeze-drying the solutions into a dried powder.

When a product in the form of a capsule is to be produced, it is preferred to further include about 2 to 15 parts by weight of a powder of lotus root joints in step b) described above, since the powder of lotus root joints contains not only active substances, but may also act as an adjuvant which is required for a capsule. The green tea extract contains mainly tea polyphenol, whereas notoginseng extract contains mainly full notoginseng saponin. In addition, a cinnamon naptha enveloped with β-cyclodextrin may also be blended into the composition of the capsule product according to the invention.

According to additional aspects of the invention, there are provided new uses for the inventive product in preparing medicines for improving human insulin resistance, for treating or preventing obesity, for treating or preventing hypertension, for treating or preventing hyperlipoidemia, for treating or preventing diabetes mellitus, for improving blood viscosity, preventing thrombosis and promoting microcirculation, and for treating or preventing Alzheimer's Disease and against senility.

The health-care products according to the invention are made from natural edible plant products, mainly from natural lotus root joints, thus taking advantages of easy availability and low prices of the raw materials, safety in administration over the long term without adverse effects, and, in addition to their capability for effectively improving IR, preventing the onset of type-II diabetes mellitus, hypertension, hyperlipoidemia and cardio-cerebral vascular diseases caused by obesity, and good anti-obesity effects.

For better understanding the essence of the present invention, the favorable medical effects of the product according to the invention are demonstrated through systemic pharmacological test data and results as follows. However, they should not be understood as any restriction to the protection scope of the present invention that is defined by the appended claims.

### EXAMPLE 1

This example demonstrates the effect of lotus root joints on a model of nutrition-type obesity rat

Materials and Test Methods

1. Preparation of Experimental Material and Feed

(a) Experimental Material

Lotus root joints were cleaned and dried in ambient air, put in an oven, and dried under forced air at 65° C. The joints were pulverized into a fine powder having a granular size of less than about 20 mesh. The powder was ready for use on the same day.

(b) Positive Medicine

The Ninghong anti-obesity tea (a product manufactured by Jiangxi Ninghong group, Trade No. 21101102) was pulverized into a fine powder for use on the same day.

(c) Experimental Feed

Concentrated high caloric feed: basal feed 40%, lard oil 35%, saccharose 15%, egg 8%, salt 2%.

Experimental feed: Samples of the concentrated high caloric feed in certain amounts were combined with certain amount of the basal feed, the powder of lotus root joints and the positive medicine powder. They were then made into model-type high caloric feed, lotus root joints-type high caloric feed and positive medicine-type high caloric feed using a pelleter. The lotus root joints-type high caloric feed contained 68% concentrated high caloric feed and 32% powder of lotus root joints. The model-type high caloric feed contained 32% basal feed place of the positive medicine powder, and the 32% of positive medicine powder in the positive medicine-type high caloric feed consisted of 21% the basal feed and 11% fine powder of the Ninghong anti-obesity tea.

After the preparation of the feeds described above, the feeds were desiccated in ambient air, and a new bath was processed about every 7 to 10 days.

2. Experimental Animals and Their Group

SD male rats having weights of 444±17 g were randomly assigned to a blank group (group T), a model control group (group II), a lotus root joints group (group III) and a positive medicine group (group IV). There were 8 animals in each group which were separately fed in a single cage.

3. Model Establishment and Administration

Each rat was allowed to have 22 g corresponding experimental feed at 5 p.m. everyday, i.e., basal feed 22 g/animal for group 1, and model-type high caloric feed, lotus root joints-type high caloric feed and positive medicine-type high caloric feed 22 g/animal were given respectively to groups, II, III, and IV (every 22 g of each feed contained 15 g concentrated high caloric feed; the remaining 7 g consist respectively of 4.6 g of basal feed, powder of lotus root joints and basal feed and 2.4 g positive medicine powder). Each rat was allowed to have 8 g basal feed the next morning, and no additional feed was additionally supplied. Tap water was available ad libitum for the rats in each group.

4. Observation Indicator and Method

(a) General items include appearance of spirit, skin and hair, eyes, activity, eating, drinking and evacuation of the animal.

(b) Intake amount: a form was designed and intake amounts were recorded every day using the form.

(c) Weights were taken once a week.

(d) Glucose tolerance was measured at the end of the 4^{th} week from the beginning of the experiment. After the rats had been fasting for 6 hours, six of them were intraperitoneally injected with 2 g glucose/Kg animal, blood was sampled through the vena caudalis, and blood sugar was measured after 0, 0.5, 1 and 2 hours using a rapid blood sugar calcimeter.

(e) Blood sugar, serum TC, TG, HDL-c and Ins on Fast: at the end of the experiment, blood was sampled from the femoral vein, the serum was separated, and a part of it was submitted to the clinical laboratory in No.1 hospital affiliated with Kunming Medical College for testing (by full automatic biochemical analyzer). Blood sugar, serum TC, TG, (color comparison in 7230 spectrophotometer) were manually measured according to kit instruction.

(f) Wet weight of celiac fat and testicle fat pad: The rat was dissected after the blood sample, the celiac fat and testicle fat pad were separated and placed them on the filtering paper to remove tissue fluid, and their weight was taken by electronic scales and record.

5. Statistical Method

The experimental data was processed with PEMS (Package for Encyclopedia of Medical Statistics, edited by Public Health-Care College of Huaxi Medical University). Single factor analysis of variance and q-test were adopted for multi-group mean and pair-wise comparison.

Test Results

1. General items. Except for the death of No. 1 rat in group II and the poor uptake of positive medicine-type feed by rats in group IV (since rats in this group had never eaten up the positive medicine-type high caloric feed given to them, making it impossible to determine the true effect of medicine, it was eliminated from the experiment), no abnormality was found in other rats.

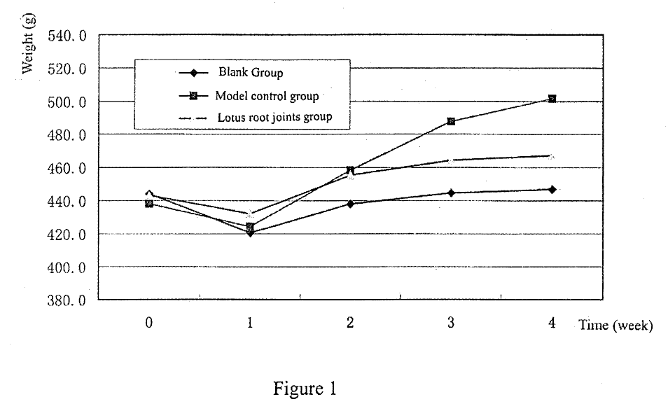

2. The changes in weight of the rats in each group at the end of the fourth week are shown in Tables 1-5 and in FIG. 1. As shown in Table 1, there was no difference in the weights of the rats in each group before the experiment, and thus the weights of each group were comparable. It was found that, at the end of the first week, there was no significant difference among the weights of rats in each group, while the rat weight of each group had decreased at various degrees. This decrease was considered to be caused mainly by the sudden change of living environment and lifestyle.

TABLE 1
Weight of rats in each group at 0^{th} week

X ± SD

| | Number of | |
|---|---|---|
| Group | animals n | Body weight |
| Blank Group | 8 | 444.0 ± 13.1 |
| Model control group | 7 | 442.0 ± 20.8 |
| Lotus root joints group | 8 | 443.0 ± 19.0 |

p > 0.05 for all

TABLE 2
Change in weight of rats in each group at the end of 1^{st} week

X ± SD

| | | | | Weight |
|---|---|---|---|---|
| | Number of | Body | Weight | increase |
| Group | animals n | weight (g) | increase (g) | rate (%) |
| Blank Group | 8 | 420.5 ± 17.0 | −23.5 ± 5.4 | −0.053 ± 0.013 |
| Model control | 7 | 424.3 ± 29.2 | −13.7 ± 14.9 | −0.032 ± 0.035 |
| group | | | | |
| Lotus root | 8 | 432.0 ± 13.5 | −11.0 ± 10.0 | −0.024 ± 0.021 |
| joints group | | | | |

| | | | |
|---|---|---|---|
| Variance | P = 0.185 | P = 0.008 | P = 0.0014 |
| analysis | | | |

Table 3 shows that at the end of the second week, the weights in the model group and the lotus root joints group were respectively 4.6% and 3.9% higher than that in the blank group, but without significant difference. According to the amount of and percentage of weight gain, the model group had the largest gain, which was significant higher than that in the blank group (P<0.05. The increase in the lotus root joints group was not as high, but still represented a significant difference from the blank group. These results indicate that the high caloric feed could increase the weight in adult rats having about a 400 g body weight, and thus continuous feeding with this kind of feed could induce obesity in rats. The preventive effect of lotus root joints on excessive weight gaining induced by high caloric feed was not demonstrated here.

TABLE 3
Change in weight of rats in each group at the end of 2^{nd} week

X ± SD

| | | | | Weight |
|---|---|---|---|---|
| | Number of | Body | Weight | increase |
| Group | animals n | weight (g) | increase (g) | rate (%) |
| Blank Group | 8 | 438.0 ± 13.8 | 17.5 ± 8.3 | 4.2 ± 2.1 |
| Model control group | 7 | 458.3 ± 28.1 | 34.0 ± 8.6* | 8.1 ± 2.2* |
| Lotus root joints | 8 | 455.3 ± 19.3 | 23.3 ± 12.9 | 5.4 ± 3.0 |
| group | | | | |

| | | | |
|---|---|---|---|
| Variance analysis | P = 0.0216 | P = 0.0211 | P = 0.0318 |

Q test, compared with blank group: *P < 0.05

Table 4 shows that at the end of the third week, the weight in the model group was significantly higher than that in the blank group (9.7% higher, p<0.01), while the lotus root joints group had no significant difference from the blank group (4.3% higher, p>0.05). According to the amplitudes of weight gain, the model group was the largest (p<0.01 compared with blank group) and the lotus root joints group was just slightly higher than blank group, but without significance. These result indicate that up-taking this high caloric feed could cause the weights of adult rats of about 400 g to be significantly heavier than that of rats at the same age who intake a common feed, and lotus root joints have a relatively significant preventive effect on the excessive weight gain caused by high caloric feed.

TABLE 4
Change in weight of rats in each group at the end of 3^{rd} week

X ± SD

| | | | | Weight |
|---|---|---|---|---|
| | Number of | Body | Weight | increase |
| Group | animals n | weight (g) | increase (g) | rate (%) |
| Blank Group | 8 | 444.5 ± 17.8 | 6.5 ± 4.6 | 1.5 ± 1.0 |
| Model | 7 | 487.7 ± 29.6** | 25.8 ± 13.4** | 6.4 ± 2.0** |
| control | | | | |
| group | | | | |
| Lotus root | 8 | 464.3 ± 19.7^{#} | 9.0 ± 3.9^{ΔΔ#} | 2.0 ± 0.8^{ΔΔ#} |
| joints group | | | | |

| | | | |
|---|---|---|---|
| Variance | P = 0.0007 | P = 0.0003 | P = 0.0000 |
| analysis | | | |

Q test, compared with blank group: **P < 0.01; compared with model control group: ^{ΔΔ}P < 0.01

Table 5 shows that at the end of the fourth week, the weight of the model group further exceeded that in the blank group (12.3% higher; p<0.01). The situation in the lotus root joints group was similar to that in the third week (4.5% higher, p>0.05), while compared with the model group, their weights had been significantly reduced (p<0.01). The amplitude of weight gain in each group was smaller than from the previous two weeks, while the amplitude in the model group was relatively larger. These results further indicate that taking-up this high caloric feed could cause the weights of adult rats of about 400 g to be significantly heavier than that of rats at the same age who intake a common feed, and lotus root joints have a significant preventive effect on the excessive weight gain caused by high caloric feed.

TABLE 5
Change in weight of rats in each group at the end of 4^{th} week

X ± SD

| | | | | Weight |
|---|---|---|---|---|
| | Number of | Body | Weight | increase |
| Group | animals n | weight (g) | increase (g) | rate (%) |
| Blank Group | 8 | 446.8 ± 15.2 | 2.2 ± 6.8 | 0.54 ± 1.59 |
| Model control | 7 | 501.7 ± 31.7** | 14.0 ± 6.2* | 2.86 ± 1.24* |
| group | | | | |
| Lotus root | 8 | 467.3 ± 23.4 | 3.0 ± 6.0^{Δ} | 0.62 ± 1.3^{Δ} |
| joints group | | | | |

| | | | |
|---|---|---|---|
| Variance | P = 0.0000 | P = 0.0117 | P = 0.032 |
| analysis | | | |

Q test, compared with blank group: *P < 0.05, **P < 0.01; compared with model control group: ^{Δ}P < 0.05

FIG. 1 is a graph of change in weight in each group during the period of the experiment from beginning to end. It can be seen from FIG. 1 that the situation of the lotus root joints group was relatively similar to that of the blank group, especially in the late stage of the experiment; the model group demonstrated continuously higher weight than the blank group. These results indicate that this high caloric feed could cause progressively excessive weight gain in adult rats of about 400 g, and the weight at the final stage of experiment was significantly higher than that of rats at the same age who intake a common feed. Lotus root joints had a relatively significant preventive effect on this weight gain trend.

3. Blood glucose (Glu), blood insulin (Ins) and insulin sensitivity index (ISI) of the rats in each group are shown in Table 6. Table 6 shows that the model group had higher Glu, but without significance, compared with the blank group; the lotus root joints group had lower Glu. Except for the lotus root joints group, the blood Ins in each experimental group was significantly higher than that in the blank group (p<0.01 for all groups), and ISI thereof was significantly lower than the blank group (p<0.01 for all groups). The ISI in the lotus root joints group had a smaller difference than the blank group (p<0.05), but was significantly higher than the other experimental groups (p<0.01 for all groups). These results indicate that this obesity model had produced hyperinsulinemia, with significant peripheral insulin resistance (IR). Further, lotus root joints could prevent the hyperinsulinemia caused by high caloric product, and could significantly reduce peripheral IR.

TABLE 6
Comparison of blood sugar, insulin (Ins),
insulin sensitivity index in each group

X ± SD

| | Number of | GLU | Ins | |
|---|---|---|---|---|
| Group | animals n | (mmol/l) | (mIU/L) | ISI |
| Blank Group | 8 | 4.55 ± | 36.98 ± | −5.108 ± |
| | | 0.58 | 6.09 | 0.1736 |
| | | | | (1.000) |
| Model control group | 7 | 5.25 ± | 68.77 ± | −5.909 ± |
| | | 0.77 | 12.09** | 0.2366** |
| | | | | (0.8645) |
| Lotus root joints group | 8 | 5.02 ± | 47.70 ± | −5.442 ± |
| | | 0.54^{#} | 23.35^{#} | 0.3068*^{ΔΔ##} |
| | | | | (0.9386) |

| | | | |
|---|---|---|---|
| Variance analysis | P = 0.0008 | P = 0.0000 | P = 0.0000 |

Q test, compared with blank group: *P < 0.05, **P < 0.01; compared with model control group: ^{ΔΔ}P < 0.01
Note:
ISI = In1/GluxIns; the numbers in parentheses are relative values.

4. The results of total cholesterol (TC), triglyceride (TG) and high density lipoprotein cholesterol (HDL-c) of rats in each group are shown in Table 7. Table 7 shows that TC in the model group was significantly higher than in the blank group (p<0.01), which was also true in the other experimental groups (p<0.01), which had no significant difference from the model group. TG in the model group was also higher, but without significant difference from the blank group. The lotus root joints group had the lowest TG. The HDL-c in the lotus root joints group was significantly higher than that in the blank group. These results indicate that this obesity model had abnormal blood fat, which was demonstrated by a significant increase of blood TC, and an increased trend of blood TG. Lotus root joints could effectively prevent the increasing trend of blood TG. While lotus root joints did not have a significant preventive effect on the increase of TC, a small effect was demonstrated. Finally, lotus root joints were shown to increase blood HDL-c.

TABLE 7
Comparison of serum TC, TG and HDL-c in each group

| | X ± SD | | | |
|---|---|---|---|---|
| | Number of | TC | TG | HDL-C |
| Group | animals n | (mmol/l) | (mmol/l) | (mmol/l) |
| Blank | 8 | 1.67 ± 0.29 | 1.62 ± 0.64 | 0.76 ± 0.12 |
| Group | | | | |
| Model | 7 | 2.44 ± 0.40** | 2.59 ± 1.21 | 0.98 ± 0.16 |
| control | | | | |
| group | | | | |
| Lotus root | 8 | 2.41 ± 0.31** | 1.54 ± 1.04^{##} | 1.10 ± 0.12** |
| joints | | | | |
| group | | | | |

| | | | |
|---|---|---|---|
| Variance | P = 0.0000 | P = 0.0007 | P = 0.0000 |
| analysis | | | |

Q test compared with blank group: **P < 0.01

5. The weight of intraperitoneal fat and the fat index in each group are shown in Table 8. Table 8 shows that the weight of intraperitoneal fat and the fat index in the model group had a significant trend to increase, whereas the lotus root joints group had a opposite trend to that of model group.

TABLE 8
Comparison of fat weight, fat index in each group

X ± SD

| | Number of | Fat | Fat |
|---|---|---|---|
| Group | animals n | weight (g) | index (g/100 g) |
| Blank Group | 8 | 10.58 ± 2.52 | 2.36 ± 0.53 |
| Model control group | 7 | 16.47 ± 5.88 | 3.27 ± 1.05 |
| Lotus root joints group | 8 | 15.30 ± 3.58 | 3.26 ± 0.66 |

| | | |
|---|---|---|
| Variance analysis | P = 0.0242 | P = 0.0652 |

Summary

1. Evaluation of obesity rat model. Usually, there are two methods for establishing an obesity model: a nutrition-type obesity model and a hypothalamic-type obesity model. The former adopts SD rats that are just past the breast, raises them with high nutritional feed (basal feed plus milk powder, egg, lard oil, vitamin AD and fresh soybean sprout, etc.) for a period of time (45 days), and their weights will significantly exceed those of rats at the same age fed with basal feed. This model adopts young rats which are in a flourishing growth period and need a lot of nutrition. Given high nutrition feed rich in protein, fat and various vitamins, they will obviously grow faster, which is demonstrated by a significant increase in weight, similar to the obesity case among children of human beings. There are no detailed reports in the literature concerning the issue of whether the obesity is complicated with other pathophysiological changes, such as abnormal blood fat, IR, glucose tolerance, etc. Analyzed from the growth phase (flourishing growth and development period) and the nutrition formulation provided (rich and complete in nutrition, high caloric), it is estimated that at least the pathological change caused by being obese in this obesity model would not be significant, which would be quite different from the practical clinical situation observed in most obese and overweight patients.

This experimental model also belongs to a nutrition-type obesity model. The rats had a weight of about 444 g, having past their flourishing growth and development period, and equivalent to an adult. The model feed adopted was basal feed plus abundant lard oil, saccharose and eggs, which contain high caloric but unbalanced nutrition, which was relatively similar to the dietary factor causing clinical obesity and related diseases such as type-II diabetes mellitus. The results of this experiment show that rat weights in the basal feed group increased slowly, while rat weights in the high caloric feed group progressively increased, and became significantly higher than that of rats in the basal feed group after a short time (28 days). These rats were complicated with significantly higher cholesterol, triglycerides and hyperinsulinemia; ISI and glucose tolerance were significantly reduced; and intraperitoneal fat and fat index also tended to increase. These results indicate that this rat obesity model could simulate most clinical adult obesity and could thus be used as a more suitable model for screening anti-obesity medicine.

2. Evaluation of lotus root joints' anti-obesity effect. In this experiment, lotus root joints were dried and pulverized into a fine powder and blended into a high caloric feed to feed three groups of rats for comparison. Meanwhile, rats fed with pure high caloric feed and basal feed were taken as the control. As a result, it was found that the weight gain in the lotus root joints group was significantly less than that of the model control group, but similar to that in the blank group, and the final weight was significantly lower than that of the model control group, without significant difference from the blank group. These results indicate that lotus root joints have a preventive effect on obesity caused by high caloric product. Its effect on celiac fat tissue was similar to described above.

Obesity is not the sole problem of weight- or fat gaining. Clinically, obesity patients often have metabolic disorders in sugar and fat. IR is an important pathophysiological basis for obesity, and also an important and harmful factor for obesity patients' susceptibility to type-II diabetes mellitus, hypertension, hyperlipoidemia and ischemic heart disease. Therefore, to evaluate the anti-obesity effect of a medicine, it should not only take weight and fat as the indicator, but should also investigate the effect of the medicine on the pathophysiological changes described above.

The results of this experiment show that the serum Ins of rats in the model control group significantly increased, while the ISI significantly decreased. Thus, a significant amount of IR had been produced, with a significant increase in serum TC. TG also showed an increasing trend. The serum Ins in the lotus root joints group was significantly lower than that of the model group, while the ISI was significantly higher than that of the model group, which indicated that IR had been relieved. Meanwhile, the serum TG level was not high. In retrospect of its effect on weight, the increasing trend of weight in lotus root joints group became similar to that in the blank group at a late stage. The above results indicate that lotus root joints had a comprehensive effect on the prevention of obesity caused by high caloric product.

In summary, the high caloric feed of this formulation could cause obesity in adult rats with complications of abnormal blood fat and IR. Lotus root joints can prevent the excessive weight gain in rats caused by this high caloric feed, and function to reduce intraperitoneal fat. Lotus root joints can also reduce IR in this obesity rat model and prevent the increase of TG thereof. Finally, lotus root joints containing relatively more starch had the best anti-obesity effect.

### EXAMPLE 2

This Example demonstrates the effect of the extract of lotus root joints on nutrition-type obesity rats.

Material and Method

1. Medicine and Preparation Thereof

Water decoction extract of lotus root joints: A proper amount of dry lotus root joints was pulverized into granules of about soybean's size. To this was added 6-10 times by weight of fresh water, based on the weight of the granules. After it was heated to boiling point, decoction was continued on small fire to keep it boiling for 30-40 minutes, and then filtered while hot. The filtrate residue was extracted once more using the same method, and the extracted solutions were combined. After being concentrated under reduced pressure and being frozen to a powder having a water content of 6%, it was sealed and stored for future use.

Ethanol refluxing extract of lotus root joints: A proper amount of dry lotus root joints was pulverized into granules of about 20 mesh. To it was added 6-10 times of 30%˜90% by weight ethanol solutions, heated in a water bath and refluxed for 1 hour, then filtered while hot. The filtrate residue was extracted once more using the same method, and the extracted solutions were combined. After concentrating the solution under reduced pressure, the ethanol was retrieved, and the material was freeze dried in vacuum to a water content of 5%, then sealed and stored for future use.

Cold maceration extract of lotus root joints: A proper amount of dry lotus root joints was pulverized into granules of about 20 mesh. To it was added 6-10 times of 30%˜90% by weight of ethanol solutions, and it was macerated at room temperature for 24 hours. After filtration, the ethanol solution at the same concentration described above was added to the filtrate residue for another 2 extractions, and the extracted solutions were combined. After concentrating the solution under reduced pressure, the ethanol was retrieved, and the material was frozen in vacuum to a powder having a water content of 5%, sealed and stored for future use.

2. Animal Group, Model Establishment and Administration of the Product.

SD male rats, weighing 434±17 g were provided by animal laboratory of Yunnan Baiyao Group Co., Ltd. The animals were randomly assigned to a blank group, a model group, a water decoction extract group (water decoction group), an ethanol refluxing extract group (ethanol refluxing group), and an ethanol cold maceration extract group (ethanol cold maceration group). There were eight animals in each group, which were fed in separate cages. The model establishment was the same as that in Example 1, i.e., basal feed was given to the blank group and high caloric feed was given to each of the other groups with the dosages the same as described before. Tap water was available ad libitum.

During the model establishment, the animals were administered with different feeds through gastric injection at 9 a.m. every day. The dosages of the three extract powders described above were 2.4 g/kg for the water decoction group, 1.92 g/kg for the ethanol refluxing group, and 1.32 g/kg for the ethanol cold maceration group. All of the powders were diluted with distilled water of the same volume before gastric injection. Distilled water of the same volume was adopted for gastric injection in the blank group and model group. The experimental period was 35 days.

3. The Observation Indicator and Method were Identical to Example 1.

4. The Statistical Method was Identical to Example 1.

Test Results

1. General items. The animals' spirit, appetite, skin and hair, and stools of the rats in each group were all as usual during the whole process of experiment.

2. The effect of the three lotus root joints extracts on the weight, Lee's index and fat index of adult nutrition-type obesity rats are shown in Table 9. Table 9 shows that at the end of the experiment, the rat's weight, Lee's index and fat index in the model group were all significantly higher than those in blank group (p<0.01). Among the three test groups, the weights of the animals in the water decoction group and the refluxing group were still heavier than those in the blank group (p<0.01 for both), while the cold maceration group had no significant difference from the blank group. Compared with the model group, the weights in the three test groups were all significantly reduced (p<0.01). Lee's index in both the water decoction group and the refluxing group was higher than in the blank group (p<0.05), while the cold maceration group had no significant difference from the blank group. Compared with the model group, Lee's index in the cold maceration group was relatively lower (p<0.01). None of the fat indices in the three test groups were significantly different from that in blank group, wherein the model group was the most similar to the blank group. These results indicate that the lotus root joints water extract, lotus root joints ethanol refluxing extract, and ethanol cold maceration extract had preventive effects on the excessive weight gain and on the increase of intraperitoneal fat in rats caused by high caloric feed. Ethanol cold maceration extract seemed to have the best effect, while lotus root joints water extract had a comparable effect to that of lotus root joints ethanol refluxing extract.

TABLE 9
Effect of three lotus root joints extracts on the weight,
Lee's index and the fat index of adult nutrition-type
obesity rats

| | X ± S | | | | |
|---|---|---|---|---|---|
| | Init. Weight | | Body length | Lee's index | Fat rate |
| Group | (g) | End weight (g) | (cm) | (g/cm²) | (g/kg) |
| Blank group | 434.5 ± 22.5 | 461.4 ± 25.6 | 25.7 ± 1.3 | 0.68 ± 0.02 | 5.78 ± 1.79 |
| Model group | 434.3 ± 19.3 | 574.2 ± 30.7** | 26.4 ± 2.1 | 0.82 ± 0.08** | 9.87 ± 2.24** |
| Water | 434.8 ± 21.2 | 530.3 ± 30.1**^{ΔΔ} | 26.3 ± 1.7 | 0.77 ± 0.04* | 8.18 ± 2.01 |
| decoction | | | | | |
| group | | | | | |
| Ethanol | 433.9 ± 15.1 | 528.3 ± 29.4**^{ΔΔ} | 26.3 ± 1.2 | 0.76 ± 0.06* | 7.92 ± 1.89 |
| refluxing | | | | | |
| group | | | | | |
| Ethanol cold | 434.7 ± 17.2 | 489.7 ± 30.0^{ΔΔ} | 26.2 ± 1.7 | 0.71 ±0.05^{ΔΔ} | 6.77 ± 1.54^{Δ} |
| maceration | | | | | |
| group | | | | | |

Compared with blank group: *p<0.05, **p<0.01;
compared with model group: ^{Δ}p<0.05, ^{ΔΔ}p<0.01;
n = 8

3. The effects of three lotus root joints extracts on the blood sugar, insulin (Ins) and ISI of adult nutrition-type obesity rats are shown in Table 10. Table 10 shows that the blood sugar in the model group was higher than that in the blank group (p<0.05), while the rest of the groups had no significant difference from the blank group. Blood Ins in the model group was significantly higher than that in blank group (p<0.01). Among the three test groups, only the water decoction group was slightly higher than the blank group (p<0.05). Compared with the model group, blood Ins in all three test groups was reduced (p<0.05, p<0.01), with the cold maceration group being the lowest (p<0.01). ISI in the model group was significantly reduced. Among three test groups, ISI in both the water decoction group and the refluxing group was reduced, while the refluxing group was even lower (p<0.01); the cold maceration group had no significant difference from the blank group. Compared with the model group, ISI in all three test groups increased to some extent, with the cold maceration group increasing the most. These results indicate that the lotus root joints water extract, lotus root joints ethanol refluxing extract, and ethanol cold maceration extract all had a preventive effect on increasing blood sugar and blood Ins in rats, and could improve IR. Ethanol cold maceration extract seemed to have the best effect, while lotus root joints water extract had a comparable effect to that of lotus root joints ethanol refluxing extract.

TABLE 10
Effect of three lotus root joints extracts on the blood sugar,
insulin (Ins) and ISI of adult nutrition-type obesity rats

| | X ± S | | |
|---|---|---|---|
| | Blood sugar | | |
| Group | (mmol/L) | Ins (mmol/L) | ISI |
| Blank group | 4.62 ± 0.51 | 36.78 ± 5.89 | □5.106 ± 0.173 |
| Model group | 5.83 ± 0.69* | 70.14 ± 12.13** | □6.013 ± 0.324** |
| Water decoction | 5.33 ± 0.84 | 56.34 ± 13.91*^{Δ} | □5.691 ± 0.412**^{Δ} |
| group | | | |
| Ethanol refluxing | 5.28 ± 0.76 | 51.48 ± 15.74^{Δ} | □5.604 ± 0.293*^{Δ} |
| group | | | |
| Ethanol cold | 5.02 ± 0.54 | 45.10 ± 14.09^{ΔΔ} | □5.416 ± 0.231^{ΔΔ} |
| maceration group | | | |

Compared with blank group: *p < 0.05, **p < 0.01; compared with model group: ^{Δ}p < 0.05, ^{ΔΔ}p < 0.01; n = 8

4. The effects of the three lotus root joints extracts on the serum TC, TG and HDL-c of adult nutrition-type obesity rats are shown in Table 11. Table 11 shows that TC in the model group significantly increased (p<0.01), as did the three test groups (p<0.01); there was no significant decrease compared with the model group. TG in the model group significantly increased (p<0.01), while the increase in the three test groups was not significant (p>0.05), and the cold maceration group was more similar to the blank group. The model group had a lower HDL-c than blank group (p<0.05). The other three groups were all higher than that of model group, with the cold maceration group being the highest (p<0.01), but all of the three groups had no significant difference from blank group. These results indicate that lotus root joints water extract, lotus root joints ethanol refluxing extract, and ethanol cold maceration extract all had no significant preventive effect on the increase of serum TC in rats caused by high caloric feed, but they did have a preventive effect on the increase of serum TG and reduction of HDL-c thereof.

TABLE 11
Effect of three lotus root joints extract on the serum
IC, TG and HDL-c of adult nutrition-type obesity rats

X ± S

| Group | IC (mmol/L) | TG (mmol/L) | HDL-c (mmol/L) |
|---|---|---|---|
| Blank group | 1.66 ± 0.30 | 1.04 ± 0.70 | 1.01 ± 0.13 |
| Model group | 2.58 ± 0.41** | 2.84 ± 1.02** | 0.74 ± 0.15* |
| Water decoction group | 2.41 ± 0.32** | 2.43 ± 1.07 | 0.98 ± 0.29^{Δ} |
| Ethanol refluxing | 2.43 ± 0.54** | 1.98 ± 1.12 | 1.06 ± 0.24^{Δ} |
| group | | | |
| Ethanol cold | 2.31 ± 0.47** | 1.68 ± 1.04 | 1.22 ± 0.26^{ΔΔ} |
| maceration group | | | |

Compared with blank group: *p < 0.05, **p < 0.01; compared with model group: ^{Δ}p < 0.05, ^{ΔΔ}p < 0.01; n = 8

Summary

Example 2 repeated the model establishment method described in Example 1, except that the period of conducting this example was extended for one week. The results showed that the weight, Lee's index and intraperitoneal fat of the animals in this model further exceeded those of normal rats, and blood sugar after a fast was higher than that of rats in the blank group. Further, there was a significant increase in blood Ins, significant reduction in ISI, significant increase in blood TC and TG, and reduction in HDL-c. All these results indicate that this nutritional rat model had significantly excellent repeatability and was a relatively ideal animal model for the study of obesity, IR and related pathophysiological disorders.

According to the experience and habit in clinical administration, Example 2 adopted several conventional extraction methods, i.e., using water decoction, 65% ethanol thermal refluxing, and 65% ethanol cold maceration to extract the total active ingredients in lotus root joints. The frozen powders prepared from the extracts were orally administered to rats and the clinical effects were compared. A control group was established using original medicinal powder. The results demonstrate that all three extracts had a preventive effect on excessive weight gain and on the increase of Lee's index and intraperitoneal fat, and some preventive effect on the increase of blood sugar after fast and on blood Ins, and could improve IR. Some preventive effect was also obtained on the increase of TG and on decrease of HDL-c. As to the increase of TC, the preventive effect of these three extracts was not significant, which was similar to that of raw lotus root joints powder.

### EXAMPLE 3

This Example determined the effect of the composition containing lotus root joints on nutrition-type obesity rats.

Material and Method

1. Medicine

(1) A freeze dried powder of cold maceration extract of lotus root joints (its preparation was identical to that recorded in Example 2

(2) notoginseng extract (notoginseng total saponin: content ≧88%, Rb1 >30%, Rg1 >20%; notoginseng leaf saponin: content of saponin element ≧32%, calculated as original ginseng diol)

(3) green tea extract (tea polyphenol ≧40%, catechin ≧25%, EGCG ≧25%, caffeine<5%)

2. Animal Group and Model Establishment Administration

SD male rats, weighing 435±16 g, were provided by the animal laboratory of Yunnan Baiyao Group Co., Ltd. Animals were randomly assigned to a blank group, a model group, a lotus root joints ethanol cold maceration extract freeze dry powder group (lotus root joints simple prescription group), a lotus root joints ethanol cold maceration extract freeze dry powder plus notoginseng extract group (lotus root joints compound prescription group 1, a lotus root joints ethanol cold maceration extract freeze dry powder plus green tea extract group (lotus root joints compound prescription group 2, and a lotus root joints ethanol cold maceration extract freeze dry powder plus notoginseng extract plus green tea extract group (lotus root joints compound prescription group 3. Eight animals were in each group, which were fed in separate cages. Basal feed was given to the blank group and high caloric feed was given to each of the other groups to establish a model (the method was identical to that in previous two Examples). Tap water was available ad libitum for each rat.

During the model establishment, feed was administered through gastric injection at 9 a.m. every day: 1.32 g/Kg lotus root joints cold maceration extract freeze dry powder for the lotus root joints simple prescription group; 1.32 g/Kg lotus root joints cold maceration extract freeze dry powder+0.066 g/Kg notoginseng saponin and 0.066 g/Kg notoginseng leaf saponin for the lotus root joints compound prescription group 1; 1.32 g/Kg lotus root joints cold maceration extract freeze dry powder+0.132 g/Kg tea polyphenol for the lotus root joints compound prescription group 2; and 1.32 g/Kg lotus root joints cold maceration extract freeze dry powder+0.066 g/Kg notoginseng saponin and 0.066 g/Kg notoginseng leaf saponin+0.132 g/Kg tea polyphenol for the lotus root joints compound prescription group 3. Each medicine described above was diluted with distilled water of the same volume before gastric injection. Distilled water of the same volume was adopted for gastric injection in the blank group and the model group. Administration of the feeds continued for 28 days.

3. Observation Indicator and Method

General Items: The animal's spirit, appetite, skin and hair, activity and stool.

The body weight, length and Lee's index: weights were taken once a week and the lengths were measured at the end of experiment (the length from nose tip to the anal of rat, expressed in cm). Lee's index=weight (g)/body length (cm²).

Glucose tolerance: After fasting for 6 hours, the rats were intraperitoneally injected with 2 g glucose/Kg, and blood sugar was measured at 0, 30, 60 and 120 minutes.

Blood sugar after fast, Ins and blood fat (including TC, TG, LDL-c and HDL-c), calculation of arteriosclerosis index AI (AI=TC/HDL-c) and Ratio of Coronary Heart Disease R-CHR (R-CHR=LDL/HDL-c).

Fat weight in body: Intraperitoneal fat, etc. converted to celiac fat g/100 g weight.

The size and amount of fat cell: A small piece of fat from the same area around genitals was fixed in 2.5% formaldehyde-ethanol solution and sliced in paraffin. The fat cells in a full visual field under 400× microscope were counted, and the size of fat cells was measured with a micrometer.

Serum SOD and LPO

Blood Viscosity

4. The Statistical Method was Identical to Example 1.

Test Result

1. General items: The general items of rats in each groups were good during the whole process of experiment, no abnormality was found.

2. The effects of lotus root joints compound prescriptions on the weight, Lee's index of nutrition-type obesity rats, are shown in Table 12. Table 12 shows that at the beginning of experiment, there was no difference in rats' weight among all of the groups. At the end of experiment, the rats' weight in the model group was significantly higher than that in the blank group (p<0.01). Among the four test groups, the simple prescription group, compound prescription group 1, and compound prescription group 2 also had weight higher than the blank group (p<0.01, P<0.05). The amplitude in the compound prescription group 2 was relatively smaller (P<0.05), but the compound prescription group 3 had no significant difference from the blank group. Compared with the model group, the weights in the four test groups were all significantly reduced (p<0.01) compared with the single prescription group; only the weight in the compound prescription group 3 was lower (p<0.05). There was no significant difference among the body length of the animals in each group. Lee's index in the model group was significantly higher than that in the blank group (p<0.01); while the four test groups had no significant difference from the blank group compared with the model group. Lee's index in the four test groups was relatively lower (p<0.01), in which the compound prescription group 3 was the lowest. These results indicate that all four of these formulations had a preventive effect on the excessive weight gain and increase of Lee's index in rats caused by high caloric feed. The compound prescription 3 had the best effect, followed by compound prescription 2, while compound prescription 1 had a comparable effect to that of a simple prescription.

TABLE 12
Effect of lotus root joints composition on the weight,
Lee's index of adult nutrition-type obesity rats

X ± S

| | | | Body | |
|---|---|---|---|---|
| | Initial | End | length | Lee's |
| Group | weight (g) | weight (g) | (cm) | index (g/cm²⁾ |
| Blank | 436.5 ± 16.2 | 458.3 ± 21.0 | 25.5 ± 1.2 | 0.69 ± 0.03 |
| group | | | | |
| Model | 436.2 ± 18.1 | 569.7 ± 28.3** | 26.1 ± 2.0 | 0.89 ± 0.05** |
| group | | | | |
| Simple | 436.8 ± 16.9 | 513.6 ± 33.4**^{ΔΔ} | 26.1 ± 1.8 | 0.75 ± 0.07^{ΔΔ} |
| pre- | | | | |
| scription | | | | |
| group | | | | |
| Com- | 436.3 ± 15.3 | 517.2 ± 30.0**^{ΔΔ} | 26.2 ± 1.3 | 0.74 ± 0.05^{ΔΔ} |
| pound pre- | | | | |
| scription | | | | |
| 1 group | | | | |
| Com- | 436.4 ± 17.2 | 503.2 ± 31.2*^{ΔΔ} | 26.1 ± 1.4 | 0.73 ± 0.06^{ΔΔ} |
| pound pre- | | | | |
| scription | | | | |
| 2 group | | | | |
| Com- | 436.5 ± 16.2 | 458.3 ± 21.0 | 25.5 ± 1.2 | 0.69 ± 0.03 |
| pound pre- | | | | |
| scription | | | | |
| 3 group | | | | |

Compared with blank group: *p < 0.05, **p < 0.01; compared with model group: ^{ΔΔ}p < 0.01; compared with simple prescription group: ^{#}p < 0.05; n = 7

3. The effects of lotus root joints compositions on the body fat and fat cells of nutrition-type obesity rats are shown in Table 13. Table 13 shows that the celiac fat of rats in the model group was significantly more than that in the blank group (p<0.01). Among the four test groups, only the simple prescription group and the compound prescription group 1 were higher than the blank group (P<0.05). The compound prescription group 3 was most similar to the blank group. Fat cells of rats in the model group were significantly less than that in the blank group (p<0.01). Among the four test groups, only the simple prescription group and the compound prescription group 1 were lower than the blank group (P<0.05, p<0.01). Compared with the model group, all four test groups had relatively more fat cells. Compared with the simple prescription group, the compound prescription group 2 and the compound prescription group 3 had more fat cells (P<0.05, p<0.01), with the compound prescription group 3 having the most and being most similar to the blank group. A fat cell diameter of rats in the model group was significantly larger than that in the blank group (p<0.01). Four test groups were also larger than the blank group, but the difference between compound prescription group 2 and compound prescription group 3 was relatively smaller (p<0.05). All four test groups had relatively smaller fat cell diameter than in the model group (p<0.01); the compound prescription group 3 had a significantly smaller diameter than the simple prescription group (P<0.01). These results indicate that all four formulations had a preventive effect on the increase of celiac fat, on fat cell hypertrophy, and on the decrease of fat cells caused by high caloric feed. Compound prescription 3 had the best effect, followed by compound prescription 2, while compound prescription 1 and the simple prescription had comparable effects.

TABLE 13
Effect of lotus root joints compound prescription on
the body fat and fat cells of nutrition-type obesity rats

X ± S

| | | Fat cell | Fat cell |
|---|---|---|---|
| Group | Celiac fat (g/kg) | (number/HPF) | size (μm) |
| Blank group | 5.27 ± 1.83 | 126.3 ± 11.5 | 27.1 ± 1.8 |
| Model group | 9.52 ± 2.11** | 85.4 ± 9.1** | 44.3 ± 2.5** |
| Simple | 8.14 ± 1.62* | 106.5 ± 10.4*^{ΔΔ} | 36.5 ± 2.1**^{ΔΔ} |
| prescription | | | |
| group | | | |
| Compound | 7.83 ± 2.23* | 98.2 ± 8.3**^{Δ} | 38.1 ± 3.4**^{ΔΔ} |
| prescription | | | |
| 1 group | | | |
| Compound | 7.07 ± 1.64 | 112.4 ± 11.5^{ΔΔ#} | 34.2 ± 3.3*^{ΔΔ} |
| prescription | | | |
| 2 group | | | |
| Compound | 6.06 ± 1.58^{Δ} | 118.5 ± 13.2^{ΔΔ##} | 31.0 ± 2.4*^{ΔΔ##} |
| prescription | | | |
| 3 group | | | |

Compared with blank group: *p < 0.05, **p < 0.01; compared with model group: ^{Δ}p < 0.05, ^{ΔΔ}p < 0.01; compared with simple prescription group: ^{#}p < 0.05, ^{##}p < 0.01; n = 7

4. The effects of the lotus root joints compound prescription on the blood fat of nutrition-type obesity rats are shown in Table 14. Table 14 shows that the serum TC of rats in the model group was significantly elevated (p<0.01). Among the four test groups, the simple prescription group was still significantly higher than the blank group (P<0.05). Compound prescription group 1 and compound prescription group 2 were slightly higher than the blank group (p<0.05). Compound prescription group 3 had no significant difference from the blank group. All three compound prescription groups were lower than the model group (p<0.05, p<0.01). Serum TG in the model group was also elevated (p<0.05). All four test groups were lower than the model group (p<0.05) with no significant difference from the blank group. LDL-c in the model group was significantly elevated (p<0.05), as was the simple prescription group (p<0.01), without an increase in other three test groups. HDL-c in the model group was reduced (p<0.05); all four test groups were higher than the model group (P<0.05), with no significant difference from the blank group. This result indicated that lotus root joints simple prescription did not have a significant preventive effect on the increase of serum TC of rats caused by high caloric feed, while compound prescription 1 and compound prescription 2 had some effect, and compound prescription 3 had the best effect. All four formulations had a preventive effect on the increase of serum TG in this model. The lotus root joints simple prescription did not have a significant preventive effect on the increase of serum LDL-c in this model, while the three compound prescriptions did. All four formulations could prevent the decrease of serum HDL-c in this model.

TABLE 14
Effect of lotus root joints compound prescription
on the blood fat of nutrition-type obesity rats

X ± S

| | TC | TG | LDL-c | HDL-c |
|---|---|---|---|---|
| Group | (mmol/L) | (mmol/L) | (mmol/L) | (mmol/L) |
| Blank | 1.54 ± 0.25 | 0.92 ± 0.64 | 2.43 ± 0.41 | 1.32 ± 0.15 |
| group | | | | |
| Model | 2.73 ± 0.41** | 2.68 ± 1.02* | 5.16 ± 1.33** | 1.08 ± 0.18* |
| group | | | | |
| Simple | 2.45 ± 0.32** | 1.34 ± 1.13^{Δ} | 4.97 ± 1.47** | 1.37 ± 0.90^{Δ} |
| pre- | | | | |
| scription | | | | |
| group | | | | |
| Com- | 2.09 ± 0.40*^{Δ} | 1.20 ± 0.89^{Δ} | 3.55 ± 1.33 | 1.32 ± 0.20^{Δ} |
| pound pre- | | | | |
| scription | | | | |
| 1 group | | | | |
| Com- | 2.14 ± 0.46*^{Δ} | 1.24 ± 0.88^{Δ} | 3.87 ± 1.54 | 1.28 ± 0.19^{Δ} |
| pound pre- | | | | |
| scription | | | | |
| 2 group | | | | |
| Com- | 1.93 ± 0.52^{ΔΔ} | 1.01 ± 0.66^{Δ} | 3.16 ± 1.39 | 1.35 ± 0.23^{Δ} |
| pound pre- | | | | |
| scription | | | | |
| 3 group | | | | |

Compared with blank group: *p < 0.05, **p < 0.01; compared with model group: ^{Δ}p < 0.05, ^{ΔΔ}p < 0.01; n = 7

5. The effects of the lotus root joints compound prescriptions on the arteriosclerosis index (AI) and ratio of coronary heart disease (R-CHD) in nutrition-type obesity rats are shown in Table 15. Table 15 shows that the AI of rats in the model group was significantly elevated (p<0.01). All four test groups had no significant difference from the blank group, wherein compound prescription group 1 and compound prescription group 3 were lower than the model group and more similar to the blank group. R-CHD in the model group was significantly higher than in the blank group (p<0.01). Among the four test groups, the simple prescription group was still significantly higher than the blank group (p<0.01). The compound prescription group 2 was slightly higher than the blank group (p<0.05). All four test groups were significantly lower than the model group (P<0.05), with three test groups being significantly lower than the simple prescription group (P<0.05, P<0.01), and compound prescription group 3 was the lowest (p<0.01). These results indicate that all four formulations had a preventive effect on the increase of AI and R-CHD in this model, wherein the compound prescription had the best result, followed by compound prescription 1 and compound prescription 2, with the simple prescription being left behind.

TABLE 15
Effect of lotus root joints compound prescription
on the AI and R-CHD of nutrition-type obesity rats

X ± S

| Group | AI | R-CHD |
|---|---|---|
| Blank group | 1.17 ± 0.37 | 1.83 ± 0.34 |
| Model group | 2.53 ± 0.75** | 4.76 ± 0.59** |
| Simple prescription group | 1.77 ± 0.81 | 3.62 ± 0.70**^{ΔΔ} |
| Compound prescription 1 group | 1.48 ± 0.69^{Δ} | 2.65 ± 0.66^{ΔΔ#} |
| Compound prescription 2 group | 1.56 ± 0.72 | 2.81 ± 0.54*^{ΔΔ#} |
| Compound prescription 3 group | 1.40 ± 0.58^{Δ} | 2.42 ± 0.45^{ΔΔ##} |

Compared with blank group: *p < 0.05, **p < 0.01; compared with model group: ^{Δ}p < 0.05, ^{ΔΔ}p < 0.01; compared with simple prescription group: ^{#}p < 0.05, ^{##}p < 0.01; n = 7

6. The effects of lotus root joints compound prescriptions on the glucose tolerance in nutrition-type obesity rats are shown in Table 16. Table 16 shows that among the fasted blood sugar of each group, the model group was relatively higher, but without significant difference. At each time point after the glucose load, the blood sugar in the model group had been significantly elevated. The blood sugars of the simple prescription group, the compound prescription group 1 and compound prescription group 2 were higher than the blank group at each time interval after the glucose load; only compound prescription group 3 was similar to the blank group. However, compared with the model group, the blood sugar of each group at each time point after the glucose load was lower (p<0.01 for all). At each time interval after the glucose load, the blood sugar in the compound prescription group 3 was lower than that in the single prescription group (p<0.01). The compound prescription group 1 and the compound prescription group 2 were lower than the single prescription group (p<0.05) at 120 minutes after the glucose load. These results indicate that all four formulations had a certain preventive effect on the decrease of glucose tolerance in rats caused by high caloric feed, among which compound prescription 3 had the best effect, compound prescription 1 and compound prescription 2 had an obvious effect and the simple prescription had a relatively weak effect.

TABLE 16
Effect of lotus root joints compound prescription on
the glucose tolerance of nutrition-type obesity rats

| X ± S | | | | |
|---|---|---|---|---|
| Group | Blood sugar (mmol/L) | | | |
| Blank group | 0 | 30 | 60 | 120 (min) |
| Model group | 5.06 ± | 11.21 ± | 9.62 ± | 5.64 ± |
| | 0.44 | 0.42 | 0.76 | 0.83 |
| Simple | 6.02 ± | 15.97 ± | 16.34 ± | 11.74 ± |
| prescription | 0.86 | 1.06** | 1.03** | 0.94** |
| group | | | | |
| Compound | 5.33 ± | 14.02 ± | 14.11 ± | 9.83 ± |
| prescription | 0.49 | 0.97**^{ΔΔ} | 1.01**^{ΔΔ} | 1.12**^{ΔΔ} |
| 1 group | | | | |
| Compound | 5.41 ± | 13.98 ± | 12.76 ± | 8.01 ± |
| prescription | 0.91 | 1.04**^{ΔΔ} | 1.21**^{ΔΔ} | 0.98**^{ΔΔ#} |
| 2 group | | | | |
| Compound | 5.30 ± | 14.11 ± | 11.22 ± | 7.88 ± |
| prescription | 0.56 | 1.15**^{ΔΔ} | 1.19**^{ΔΔ} | 1.02**^{ΔΔ#} |
| 3 group | | | | |

Compared with blank group: *p < 0.05, **p < 0.01; compared with model group: ^{Δ}p < 0.05, ^{ΔΔ}p < 0.01; compared with simple prescription group: ^{#}p < 0.05, ^{##}p < 0.01; n = 7

7. The effects of the lotus root joints compound prescriptions on the blood sugar, Ins and ISI in nutrition-type obesity rats are shown in Table 17. Table 17 shows that there was no significant difference among the fasted blood sugar of each group, while the fasted serum Ins in the model group significantly increased (P<0.01). All four test groups had significantly lower serum Ins than the model group (P<0.01), with no significant difference from the blank group. Ins in the model group was significantly lower than in the blank group (P<0.01). Among the four test groups, the simple prescription group, compound prescription group 1 and compound prescription group 2 were still lower than the blank group (P<0.05). The compound prescription group 3 was similar to the blank group. All four test groups were significantly higher than the model group. These results indicate that all four formulations could prevent the increase of serum Ins caused by high caloric feed, thus reducing the decrease of the body's sensitivity to insulin, and compound prescription 3 had the best effect, while the other three prescriptions had similar effects.

TABLE 17
Effect of lotus root joints compound prescription on the
blood sugar, Ins and ISI of nutrition-type obesity rats

X ± S

| | Blood sugar | | |
|---|---|---|---|
| Group | (mmol/L) | Ins (Mu/L) | ISI |
| Blank group | 4.38 ± 0.52 | 36.54 ± 5.78 | −5.142 ± 0.164 |
| Model group | 5.44 ± 0.76 | 69.08 ± 11.33** | −6.083 ± 0.214** |
| Simple | 5.03 ± 0.48 | 48.14 ± 15.22^{ΔΔ} | −5.504 ± 0.204*^{ΔΔ} |
| prescription | | | |
| group | | | |
| Compound | 5.21 ± 0.83 | 46.57 ± 12.36^{ΔΔ} | −5.493 ± 0.291*^{ΔΔ} |
| prescription | | | |
| 1 group | | | |
| Compound | 5.13 ± 0.72 | 47.03 ± 13.34^{ΔΔ} | −5.481 ± 0.128*^{ΔΔ} |
| prescription | | | |
| 2 group | | | |
| Compound | 4.79 ± 0.55 | 42.35 ± 7.83^{ΔΔ} | −5.301 ± 0.172^{ΔΔ} |
| prescription | | | |
| 3 group | | | |

Compared with blank group: *p < 0.05, **p < 0.01; compared with model group: ^{Δ}p < 0.05, ^{ΔΔ}p < 0.01; n = 7

8. The effects of the lotus root joints compound prescriptions on the blood viscosity in nutrition-type obesity rats are shown in Table 18. Table 18 shows that serum viscosity in the model group significantly increased (P<0.01). All four test groups had lower serum viscosity than the model group, in which the three compound prescription groups were even lower (P<0.01). The simple prescription group and the compound prescription group 2 were still higher than the blank group (P<0.01, P<0.05). Both the compound prescription group 1 and the compound prescription group 3 were significantly lower than the simple prescription group. The Hct in the model group was larger than in the blank group (P<0.05). While the four test groups had no significant difference from the blank group, compound prescription group 3 was most similar to the blank group. These results indicate that all four formulations had a preventive effect on the increase of serum viscosity in this model. Compound prescription 3 and compound prescription 1 had the best effects, followed by compound prescription 2, with the simple prescription having a relatively weak effect. The situations were similar with regard to their effect on Hct.

TABLE 18
Effect of lotus root joints compound prescription on
the blood viscosity of nutrition-type obesity rats

X ±S

| | Serum | |
|---|---|---|
| Group | viscosity (Cp) | Hct (%) |
| Blank group | 1.32 ± 0.09 | 34.12 ± 2.01 |
| Model group | 2.26 ± 0.17** | 37.85 ± 1.98* |
| Simple prescription group | 1.98 ± 0.34**^{Δ} | 35.87 ± 2.15 |
| Compound prescription 1 group | 1.35 ± 0.28^{ΔΔ##} | 34.35 ± 1.79 |
| Compound prescription 2 group | 1.77 ± 0.21*^{ΔΔ} | 35.76 ± 2.43 |
| Compound prescription 3 group | 1.34 ± 0.31*^{ΔΔ##} | 34.19 ± 2.03^{Δ} |

Compared with blank group: *p < 0.05, **p < 0.01; compared with model group: ^{Δ}p < 0.05, ^{ΔΔ}p < 0.01; compared with simple prescription group: ^{#}^{#}p < 0.01; n = 7

9. The effects of the lotus root joints compound prescriptions on SOD and LPO in nutrition-type obesity rats are shown in Table 19. Table 19 shows that serum SOD in the model group significantly decreased (P<0.01), with no significant decrease in the four test groups; compound prescription group 3 and compound prescription group 2 were more similar to blank group. LPO in the model group significantly increased (P<0.01). All four test groups were significantly lower than that of the model group (P<0.01). With the exception that the simple prescription group was still higher than blank group (P<0.01), all three compound prescription groups had no significant difference from blank group. Compound prescription group 3 was most similar to the blank group, followed by compound prescription group 2. All three compound prescription groups were lower than the simple prescription group (P<0.05, p<0.01), in which compound prescription group 2 and compound prescription group 3 were even lower (p<0.01). These results indicate that all four formulations had a preventive effect on the increase of serum SOD and LPO in this model. Compound prescription 3 and compound prescription 2 had the best effect, followed by compound prescription 1, while the simple prescription had a relatively weak effect.

TABLE 19
Effect of lotus root joints compound prescription
on the SOD and LPO of nutrition-type obesity rats

X ± S

| Group | SOD (u/g) | LPO (nmol/ml) |
|---|---|---|
| Blank group | 17.31 ± 1.94 | 0.69 ± 0.12 |
| Model group | 12.35 ± 2.01** | 2.74 ± 0.45** |
| Simple prescription group | 14.83 ± 1.99^{Δ} | 1.58 ± 0.37**^{ΔΔ} |
| Compound prescription 1 group | 15.02 ± 2.35 | 1.16 ± 0.23^{ΔΔ#} |
| Compound prescription 2 group | 16.04 ± 2.51^{Δ} | 0.98 ± 0.32^{ΔΔ##} |
| Compound prescription 3 group | 16.98 ± 1.84^{ΔΔ} | 0.81 ± 0.14^{ΔΔ##} |

Compared with blank group: *p < 0.05, **p < 0.01; compared with model group: ^{Δ}p < 0.05, ^{ΔΔ}p < 0.01; compared with simple prescription group: ^{#}p < 0.05, ^{##}p < 0.01; n = 7

Summary

Example 3 adopted the adult nutrition-type obesity rat model which had been proven to have good repeatability. Investigations were made on the effects of pure lotus root joints product and lotus root joints combined with extracted total notoginseng saponin and green tea extract tea polyphenol on many items of animals in this model such as weight, celiac fat, fat cell size and number, glucose tolerance, IR, blood fat, blood viscosity and LPO, etc. The results showed that using the pure lotus root joints product caused certain improvements on items described above except serum TC, but its effect on celiac fat reduction, blood fat adjustment, blood viscosity improvement, anti-arteriosclerosis, anti-oxidization was still weak. In combination with total notoginseng saponin, it could cause more significant decrease in serum TC and blood viscosity, and cause quite smaller AI and R-CHD. In combination with tea polyphenol, it could cause the amount of celiac fat and the number and size of fat cells to be closer to normal value, and could increase the activity of SOD, reduce LPO. If it is combined with notoginseng total saponin and tea polyphenol, then all the above items would tend to become normal.

Notoginseng and one of its main effective active ingredients, notoginseng total saponin, is a Chinese traditional medicine and natural chemical substance under extensive study. Many studies have shown that notoginseng saponin and notoginseng leaf saponin have good effect on blood fat adjustment, blood viscosity reduction and protection of vascular endothelial function. Obesity patients often show a complication of hyperlipoidemia and increased blood viscosity, with significantly higher incidence of arteriosclerosis than persons without obesity. This experiment adopted lotus root joints in combination with notoginseng saponin and notoginseng leaf saponin. The results indicate that the blood fat and blood viscosity in this obesity model tend to become normal, thus indicating that this combination was necessary for the comprehensive treatment of obesity.

Green tea is an herbal drug/food double function product that has been used for weight and fat reduction for thousands of years; one of its main active ingredients, tea polyphenol, is also a kind of natural chemical substance under extensive study. Studies have shown that tea polyphenol has a significant inhibiting effect on the accumulation of fat in the body and liver of rats which have been raised with fat-rich feed, and it could reduce TC in blood. At the same time, tea phenol also has excellent anti-oxidization effects and could inhibit the oxidization of cholesterol through self-oxidization, thus reducing the deposition of cholesterol on artery walls. The use of lotus root joints in combination with tea polyphenol brought about this result: celiac fat in this model had significantly decreased, with significant decrease in fat cell's size. At the same time, blood SOD significantly increased, with significant reduction of LPO, thus leading to the conclusion that this composition had a comprehensive and good effect on obesity and the effect was also better than the single use of lotus root joints.

Compound prescription 3 in this experiment adopted lotus root joints extract as the principal drug to be combined with saponin from root, stem and leaf of notoginseng and with green tea polyphenol. The results showed that this combination had integrated the advantages of these three substances; this model had achieved a relatively ideal effect in every respect. It was indicated that lotus root joints as the principal drug, in combination with saponin from root, stem and leaf of notoginseng and with green tea polyphenol, was a relatively ideal combination for the treatment of obesity.

Obesity is a complicated disease with various conditions. It is not only a matter of gaining in weight and fat, but, more importantly, obesity patients have significant IR and glucose and fat metabolic disorders. It is the very pathophysiological disorders that complicate obesity patients with other serious diseases, such as type-IT diabetes mellitus, abnormal blood fat, hypertension and arteriosclerosis, and cause the average lifetime of the obesity population to be shorter than that of the non-obesity population. The new combination according to the invention uses lotus root joints as the principal active ingredient. It not only has good anti-obesity effect, but more importantly, it also has an outstanding effect on significantly improving IR. IR is the pathophysiological basis for obesity patient to have metabolic disorders in glucose, fat and to be susceptible for these serious diseases. Therefore, lotus root joints products, such as its powder or its extract, used as the principal active ingredient in the prescription, have an irreplaceably important effect on the treatment and prevention of obesity. A combination with tea polyphenol and notoginseng saponin obtained from root, stem, and leaf makes the prescription more effective, act more quickly for cleaning unwanted fat within body, for adjusting blood fat, improving of blood viscosity and protecting arterial walls, etc. Further, it makes the prescription capable of not only hitting the key point of obesity, i.e., IR, enabling a thorough treatment of the basic grounds causing obesity, but also takes advantage of a combination of various positive effects on various pathophysiological disorders in obesity. Thus, the product of the invention has better a effect not only on obesity itself, but also on the treatment and prevention of various complications thereof.

Beneficial Effect

In one word, the present invention represents a substantial breakthrough in the development of a safe and effective health-care product that could comprehensively improve IR and its related diseases through extensive and systemic study. The present invention adopts natural lotus root joints as the principal raw material, which is abundant in source, without adverse effects, of high quality and inexpensive, and readily acceptable to people, and thus could benefit millions of patients. The lotus root joints composition according to the invention in combination with tea polyphenol and notoginseng root-, stem- and leaf saponin, makes the prescription more effective, acts more quickly for cleaning unwanted fat within body, for adjusting blood fat, for improving blood viscosity and for protecting arterial walls, etc. The product of the invention has not only a good therapy effect on the key link of being obesity, i.e., IR, enabling an effective and thorough treatment of it, but could also take the advantage of the combination of various positive effects of the various active ingredients, taking account of various complicated pathophysiological disorders caused from being obesity. Thus, this combination has surprising effect not only on obesity itself, but also on the treatment and prevention of type-II diabetes mellitus, hypertension, hyperlipoidemia and cardio-cerebral vascular diseases induced by it.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

We claim:

1. A health-care product, comprising at least one of a powder and an extract of lotus root joints.

2. The health-care product according to claim 1, comprising about 4 to 30 parts by weight of the extract of lotus root joints, about 0.08 to 2 parts by weight of a green tea and/or an extract thereof; and about 0.08 to 0.5 parts by weight of notoginseng and/or an extract thereof.

3. The health-care product according to claim 2, comprising about 4 to 7.5 parts by weight of the extract of lotus root joints.

4. The health-care product according to claim 2, further comprising about 2 to 15 parts by weight of the powder of lotus root joints.

5. The health-care product according to claim 1, wherein the product is in a form selected from the group consisting of a tablet, a capsule, a soluble granule, a solution and an injection.

6. A method for preparing a health-care product, comprising the steps of:
a) Pulverizing lotus root joints to obtain a powder of the joints;
b) Putting the powder of the joints in a product-grade solvent to form a mixture for extraction, filtering the mixture to form a filtrate solution, and using the filtrate solution as an extract of the joints; and
c) Blending at least one conventional product-grade adjuvant with at least one of the powder of the joints and the extract of the joints, and formulating the blend into a form selected from the group consisting of a tablet, a capsule, a soluble granule, a solution and an injection.

7. The method according to claim 6, wherein steps a) and b) comprise:
(1) Drying lotus root joints and pulverizing the dried joints into a powder having a granule size of about 20 mesh;
(2) Adding to the powder about 5 to 10 times by weight of about 30%˜90% ethanol based on a weight of the powder to form a mixture;
(3) Extracting the mixture at room temperature for about 24 hours and filtering the mixture to produce a filtrate solution and a residue;
(4) Repeating step (3) at least two additional times;
(5) Combining the filtrate solutions from steps (3) and (4); and
(6) Concentrating the combined filtrate solution under vacuum and freeze-drying the solution into a dried powder.

8. The method according to claim 6, wherein steps a) and b) comprise:
(1) Drying lotus root joints and pulverizing the dried joints into a powder having a granule size of about 20 mesh;
(2) Adding to the powder about 5 to 10 times by weight of about 30%˜90% ethanol based on a weight of the powder to form a mixture;
(3) Heating the mixture in a water bath, refluxing the mixture for about 30 to 60 minutes, and filtering the hot mixture to produce a filtrate solution and a residue;
(4) Repeating step (3) at least two additional times;
(5) Combining the filtrate solutions from steps (3) and (4); and
(6) Concentrating the combined filtrate solution at a low temperature under a reduced pressure and freeze-drying the solution into a dried powder.

9. The method according to claim 6, wherein steps a) and b) comprise:
(1) Drying lotus root joints and pulverizing the dried joints into granules having about a size of a soybean;
(2) Adding to the granules about 6 to 10 times by weight of fresh water based on a weight of the granules to form a mixture;
(3) Heating the mixture at a boiling point for about 30 to 40 minutes and filtering the hot mixture to produce a filtrate solution and a residue;
(4) Repeating step (3) at least two additional times;
(5) Combining the filtrate solutions from steps (3) and (4); and
(6) Concentrating the combined filtrate solution under vacuum and freeze-drying the solution into a dried powder.

10. The method according to claim 6, wherein the method comprises performing steps (a) and (b) to prepare about 4 to 7.5 parts by weight of the extract of lotus roots joints, further comprising adding about 0.08 to 2 parts by weight of a green tea and/or an extract thereof and about 0.08 to 0.5 parts by weight of notoginseng and/or an extract thereof.

11. The method according to claim 10, further comprising adding about 2 to 15 parts by weight of the powder of lotus root joints.

12. A method for preparing a medicine selected from the group consisting of a medicine for improving human insulin resistance; a medicine for treating or preventing obesity; a medicine for treating or preventing hypertension; a medicine for treating or preventing hyperlipoidemia; a medicine for treating or preventing diabetes mellitus; a medicine for improving blood viscosity, preventing thrombosis, and promoting microcirculation; and a medicine for treating or preventing Alzheimer's disease and senility; the method comprising using the health care product according to claim 1 to form the medicine.
